(19) United States
(12) Patent Application Publication
(10) Pub. No.: US 2016/0067472 A1
(43) Pub. Date: Mar. 10, 2016

(54) CATHETER ADAPTER APPARATUS

(71) Applicant: Covidien LP, Mansfield, MA (US)

(72) Inventors: Brett Haarala, Framingham, MA (US); Richard Braga, North Easton, MA (US)

(21) Appl. No.: 14/877,975

(22) Filed: Oct. 8, 2015

Related U.S. Application Data
(62) Division of application No. 12/041,447, filed on Mar. 3, 2008, now abandoned.
(60) Provisional application No. 60/904,484, filed on Mar. 2, 2007.

(51) Int. Cl. A61M 39/10; A61M 1/34

(52) U.S. Cl. CPC A61M 39/10; A61M 1/34; A61M 2039/1077

(57) ABSTRACT

A catheter adapter apparatus includes an adapter body. The adapter body includes a fluid source end adapted for connection to a fluid source and having an internal bore to transfer the fluids supplied by the fluid source, and a catheter end for connection to a catheter tube. The catheter end includes first and second members. Each of the first and second members is adapted for reception within corresponding lumens of the catheter tube. At least the first member defines an internal bore in fluid communication with the fluid source end to transfer the fluids supplied by the fluid source.

### CROSS-REFERENCE TO RELATED APPLICATION(S)

This application is a divisional of U.S. application Ser. No. 12/041,447, filed Mar. 3, 2008, which claims priority to and the benefit of U.S. Provisional Application Ser. No. 60/904,484, filed Mar. 2, 2007, the entire content of each of which is incorporated by reference herein.

### BACKGROUND

1. Technical Field

The present disclosure relates to a catheter adapter and, more particularly, relates to a catheter adapter for use with a hemodialysis catheter to facilitate the performance of priming, flushing and aspiration functions in connection with a hemodialysis procedure.

2. Background of Related Art

Catheters are flexible medical instruments intended for the withdrawal and introduction of fluids relative to body cavities, ducts, and vessels. Catheter instrumentation may have particular application in a hemodialysis procedure where blood is withdrawn from a blood vessel for treatment and subsequently returned to the blood vessel for circulation. Known hemodialysis catheters include multiple lumens, such as dual lumen or triple-lumen catheters, permitting bi-directional fluid flow within the catheter whereby one lumen is dedicated for withdrawal of blood and the other lumen is dedicated for returning the treated blood to the vessel. During an exemplary hemodialysis procedure, a multiple lumen catheter is inserted into a body and blood is withdrawn through an arterial lumen of the catheter. The removed blood is directed to a hemodialysis unit, which dialyzes or purifies the blood to remove waste and toxins. The dialyzed blood is returned to the patient through a venous lumen of the catheter.

Various techniques are employed for the insertion of hemodialysis catheters including, e.g., with the use of guidewires, introduction sty lets or the like. Some of these known techniques include subcutaneous tunneling methodologies, including ante grade and reverse tunneling techniques, where a subcutaneous tunnel is formed between two spaced openings in the skin with the use of a trocar or the like. One end of the catheter may be introduced into, e.g., the jugular vein, and routed to the heart. The remaining end of the catheter may be attached to a trocar and pulled through the tunnel to expose this catheter end from one of the openings. The remaining exposed catheter end is fluidly connected to a hemodialysis machine.

### SUMMARY

Accordingly, the present disclosure is directed to a catheter adapter apparatus for connection to a catheter to facilitate aspiration and/or irrigation of the catheter lumens, and possibly priming the lumens to receive fluid under pressure. The catheter adapter apparatus has one application in a hemodialysis procedure where the catheter is positioned via a subcutaneous tunneling technique. In connection with this technique, the catheter lumens of the catheter may require flushing and/or aspiration to clear the catheter lumens of foreign tissue debris. In addition, the catheter lumens may require priming to permit the pumping mechanism of the hemodialysis machine to properly operate.

In one embodiment, the catheter adapter apparatus includes an adapter body. The adapter body includes a fluid source end adapted for connection to a fluid source and having an internal bore to transfer the fluids supplied by the fluid source, and a catheter end for connection to a catheter tube. The catheter end includes first and second members. Each of the first and second members is adapted for reception within corresponding lumens of the catheter tube. At least the first member defines an internal bore in fluid communication with the fluid source end to transfer the fluids supplied by the fluid source. The second member may define an internal bore in fluid communication with the internal bore of the fluid source end. The catheter adapter apparatus may further include a valve member associated with the adapter body. The valve member may be movable to selectively fluidly connect the internal bores of the first and second members with the internal bore of the fluid source end. The fluid source end of the adapter body may include a luer connector.

### BRIEF DESCRIPTION OF THE DRAWINGS

Embodiments of the presently disclosed catheter adapter apparatus are described herein with reference to the accompanying drawings, wherein:

FIG. 1 is a perspective view of a catheter adapter apparatus constructed in accordance with one embodiment of the present disclosure;

FIG. 2A is a perspective cross-sectional view of the catheter adapter apparatus of FIG. 1 illustrating the valve of the catheter adapter apparatus in a first position;

FIG. 2B is a perspective cross-sectional view of the catheter adapter apparatus of FIG. 1 illustrating the valve in a second position;

FIG. 2C is a perspective cross-sectional view of the catheter adapter apparatus of FIG. 1 illustrating the valve in a third position;

FIG. 2D is a perspective cross-sectional view of the catheter adapter apparatus of FIG. 1 illustrating the valve in a fourth position;

FIG. 2E is a side cross-sectional view of the catheter adapter apparatus of FIG. 1 incorporating a slit valve;

FIG. 2F is a perspective view of the catheter adapter apparatus of FIG. 1 connected to a catheter and receiving a guide wire;

FIG. 3 is a perspective view of a catheter adapter apparatus in accordance with another embodiment of the present disclosure;

FIG. 4 is a perspective cross-sectional view of the catheter adapter apparatus of FIG. 3;

FIGS. 5A-5B are views illustrating one tunneling procedure for implanting a hemodialysis catheter;

FIG. 5C-5D are views illustrating an alternate tunneling procedure for implanting a hemodialysis catheter;

FIG. 5E is a view illustrating the catheter implanted in accordance with either tunneling procedure and the trailing end of the catheter exposed for fluid connection to a hemodialysis machine;

FIG. 6 is a front cross-sectional view of a dual lumen catheter illustrating the catheter adapter apparatus mounted within the catheter;

FIG. 7 is a flow chart illustrating the method of using the catheter adapter of FIG. 1; and

FIG. 8 is a view of a template for use with the tunneling procedure.

### DETAILED DESCRIPTION OF THE EMBODIMENTS

Embodiments of the presently disclosed catheter adapter apparatus will now be described in detail with reference to the drawing figures wherein like reference numerals identify similar or identical elements.

In the discussion that follows, the term “proximal” or “trailing” will refer to the portion of a structure that is closer to a clinician, while the term “distal” or “leading” will refer to the portion that is farther from the clinician. As used herein, the term “subject” refers to a human patient or other animal. The term “clinician” refers to a doctor, nurse or other care provider and may include support personnel.

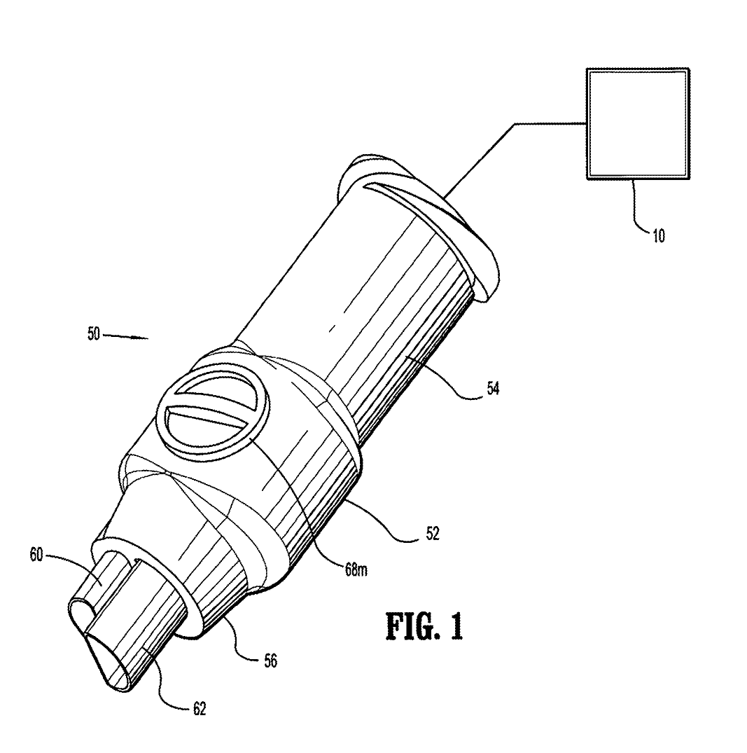

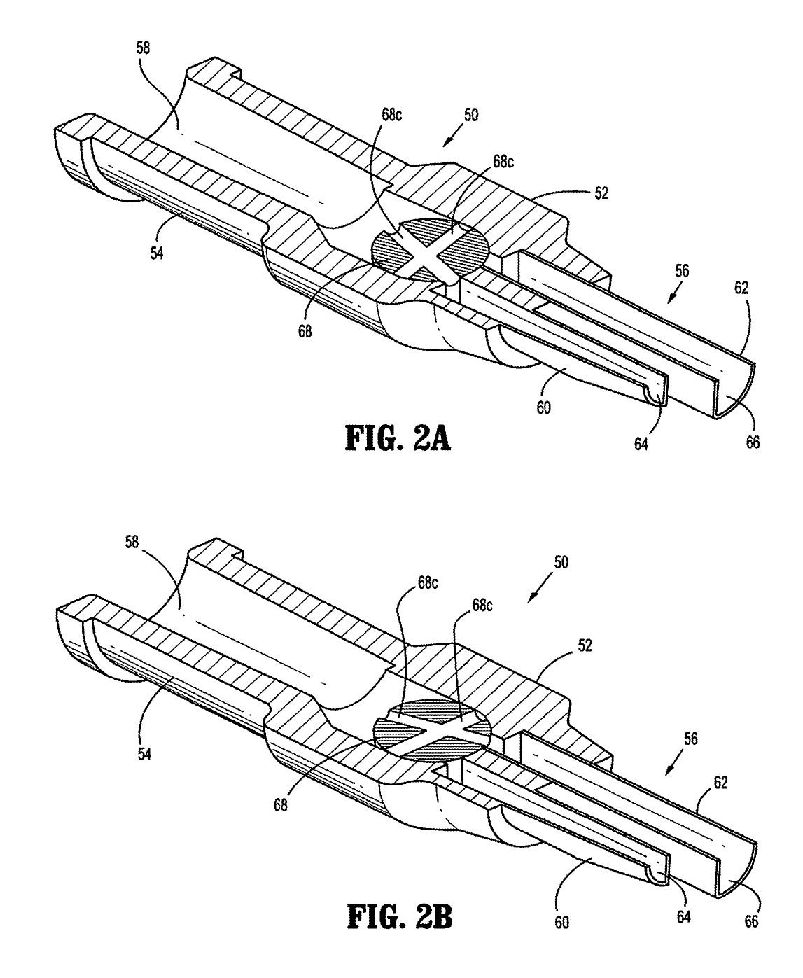

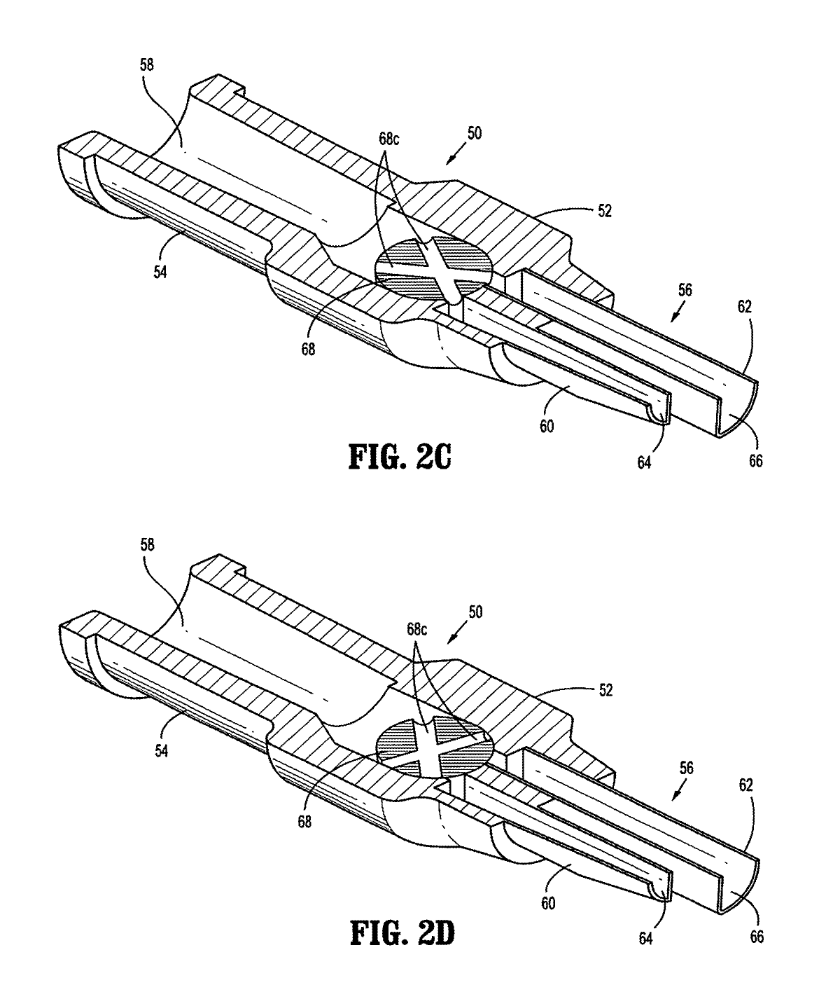

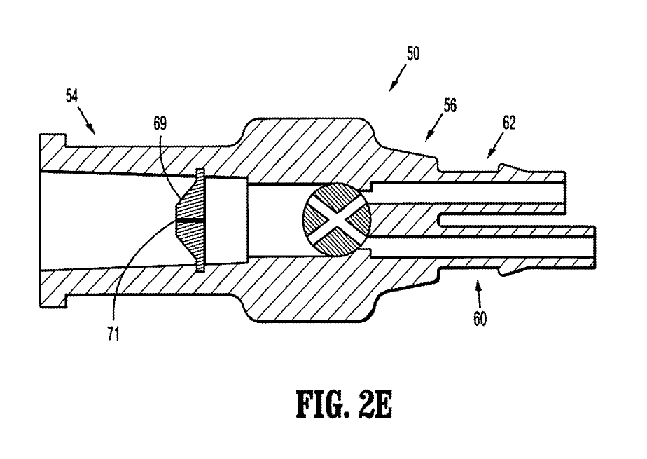

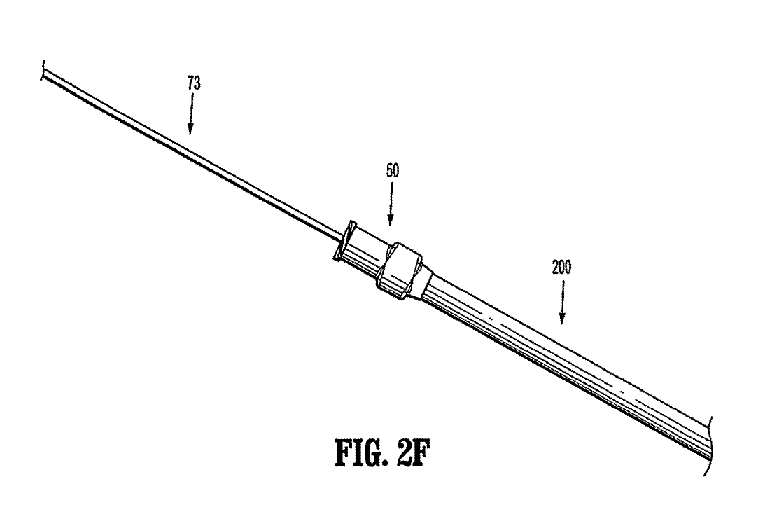

Referring now in detail to FIGS. 1 and 2A, there is illustrated a catheter adapter apparatus in accordance with an embodiment of the disclosure, and generally designated as reference numeral 50. Catheter adapter 50 includes adapter body 52 having fluid source end 54 adapted for connection to a fluid source 10 shown schematically in FIG. 1, and catheter end 56 adapted for connection to a catheter tube. Fluid source end 54 may include a luer-type fitting, although any other suitable fluid-tight fittings may substitute the luer-type fitting. Fluid source end 54 is adapted for fluid-tight engagement with the fluid source 10. Fluid source end 54 defines internal bore 58, which may be is in fluid communication with the fluid source 10 upon connection thereto. Fluid source 10 may be a syringe or any other mechanical source of vacuum or irrigation fluids suitable for priming, flushing and/or aspiration functions.

Catheter end 56 includes first and second members 60, 62 extending from adapter body 52. First and second members 60, 62 are adapted for reception within corresponding lumens (e.g., outflow and inflow lumens of a hemodialysis catheter) of a catheter tube. In one embodiment, first and second members are generally D-shaped in cross-section for reception within corresponding D-shaped lumens of a catheter tube. Other shapes are also envisioned including rounded, oval, rectangular or the like. First and second members 60, 62 each define internal lumens 64, 66 extending completely therethrough and in fluid communication with internal bore 58 of fluid source end 54 of adapter body 52.

In one alternate embodiment, adapter body 52 includes a valve mechanism to selectively fluidly couple internal lumens 64, 66 of first and second members 60, 62 with internal bore 58 of fluid source end 54. The valve mechanism may include a gate valve disposed within adapter body 52 and movable to open one internal lumen 64, 66 while closing the other internal lumen 64, 66. In an alternate embodiment, the valve mechanism incorporates a ball valve 68 as shown in FIG. 2A with at least one and possibly two internal passages 68c, 68d. The ball valve 68 may be movable to selectively establish fluid communication with either or both internal lumens 64, 66 with internal bore 58 of fluid source end 54 and the fluid source, or, optionally, prevent fluid communication between the components. Other arrangements or suitable directional control valves are also envisioned.

The valve mechanism may also include a manually manipulative actuator identified schematically in FIG. 1 as reference numeral 70, and operatively coupled to any valve 68 (e.g., a gate valve, a ball valve, or a check valve) in adapter body 52. Any mechanism to connect actuator 70 to valve 68 is envisioned. Actuator 70 is movable to selectively arrange valve 68 at a variety of positions. For example, actuator 70 may be selectively movable between at least two positions. In a first position, actuator 70 positions the valve 68 to fluidly couple internal lumen 64 of first member 60 with internal bore 58 via passage 68c and prevent flow between internal lumen 66 and the internal bore 58, as illustrated in FIG. 2A. In a second position of actuator 68m, the valve is repositioned and fluidly couples only internal lumen 66 of second member 62 with passage 68d, while preventing flow to and from internal lumen 64 of first member 60, as depicted in FIG. 2B. As seen in FIG. 2C, a third position is also contemplated. In the third position, actuator 68m may reposition valve such that each of the lumens 64, 66 of first and second members 60, 62 are in fluid communication with internal bore 58 of fluid source end 54 through passages 68c, 68d. Actuator 70 may also move valve 68 to a fourth position, as illustrated in FIG. 4D. In the fourth position, the ball valve prevents fluid communication between each of the lumens 64, 66 of first and second members 60, 62 and the internal bore 58 of the fluid source end 54. One suitable ball valve mechanism which may be incorporated into the catheter adapter 50 of the present disclosure is disclosed in U.S. Pat. No. 5,395,342 to Yoon, the entire contents of which is incorporated herein by reference. Actuator 70 may further include a visual indicator 72 adapted to visually designate the position of valve. Visual indicator 72 may alternatively show the clinician the direction of the movement necessary to place valve 68 in the first, second, or third position.

In use, catheter adapter 50 is connected via fluid source end 54 of adapter body 52 to a source of pressure, vacuum or irrigation fluid (not shown) and fluidly couples the fluid source to a catheter. The catheter may have one, two or more lumens. When coupled to a dual lumen catheter, catheter adapter 50 allows the clinician to selectively apply pressure, vacuum or irrigation fluid to the lumens of the dual lumen catheter. In one application, the catheter may be a dual lumen catheter including two generally D-shaped lumens separated by septum wall. For example, suitable catheters include the catheter extrusion or tube incorporated in the Mahurkar® dual and triple lumen catheters available from Covidien. The catheter may be fabricated from a suitable elastomeric, thermoplastic or polymeric material, and manufacturing through known extrusion or molding techniques or any other conventionally acceptable methodology. When catheter adapter 50 is connected to a dual lumen catheter, the clinician applies pressure/vacuum or irrigation fluid to catheter adapter 50 through internal bore 58. By operating actuator 70, the clinician selects which of lumens 64, 66 of first and second members 60, 62 is fluidly coupled to the pressure/vacuum/irrigation source. Since first and second members 60, 62 are coupled to separate lumens of the dual lumen catheter, the clinician may flush or aspirate one lumen at a time. Alternately, the clinician may operate actuator 70 such that both lumens 64, 66 are fluidly coupled to the pressure/vacuum source, thereby allowing flushing or aspiration of both the lumens 64, 66 simultaneously.

It is also contemplated that one or more one-way valves, check valves, or the like may be included in catheter adapter 50 or externally connected thereto. These one-way valves permit fluid to be introduced into an attached dual lumen catheter or other connected device and inhibit reverse flow from the dual lumen catheter or connected device towards the pressure/vacuum source. In an alternative embodiment, catheter adapter 50 may incorporate a stopcock valve or any other suitable valve for allowing or occluding fluid flow in both directions.

With reference to FIG. 2E, catheter adapter 50 may alternatively incorporate slit valve 69. Slit valve 69 may be made of an elastomeric material and includes a slit 71 defining a passage adapted to receive a surgical instrument such as a guidewire or needle. Slit 71 may be adapted to open in the presence of the guidewire and form a substantial fluid tight seal with the guidewire, and substantially close in the absence of the guidewire. In the closed position, slit valve 69 minimizes the passage of fluids such as air or saline. Other valve types are also envisioned including a zero closure valve such as a duckbill valve, septum valve or any other commercially available passive or directional valves or the like.

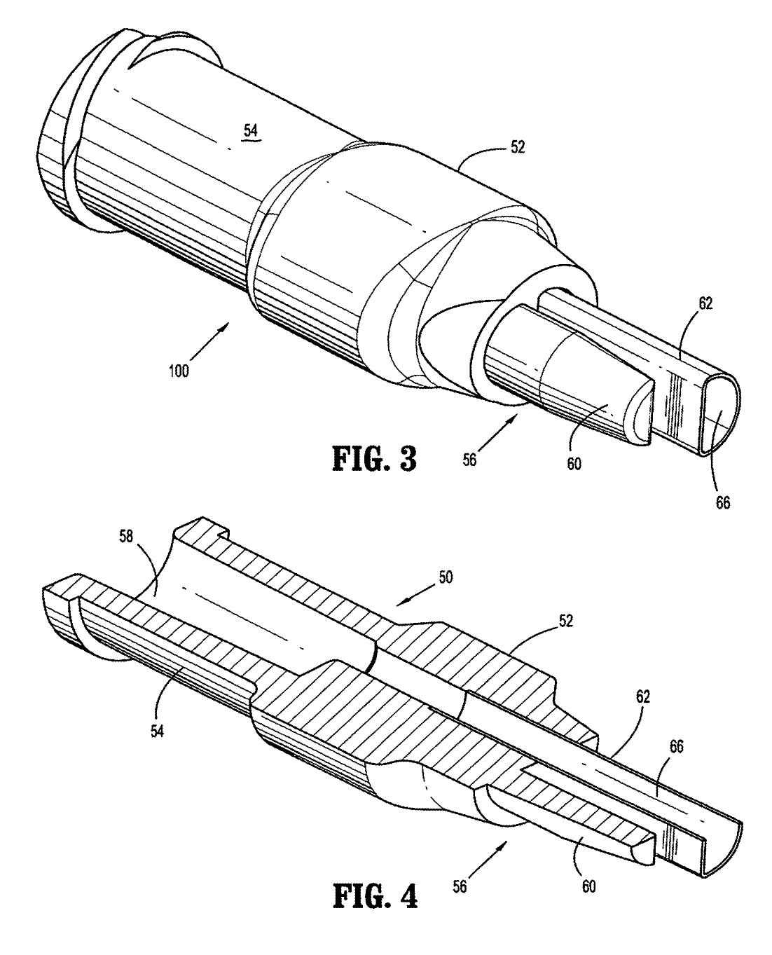

FIGS. 3 and 4 illustrate an alternate embodiment of catheter adapter 100. Catheter adapter 100 is substantially similar to catheter adapter 50 of FIG. 1; however, in accordance with this embodiment, first member 60 is devoid of an internal lumen, i.e., the first member 60 is solid. In use, fluid source end 54 of adapter body 52 is connected to a source of fluid or vacuum (not shown) and catheter end 56 of the adapter body 52 is connected to the catheter tube by insertion of first and second members 60, 62 within corresponding catheter lumens of the catheter tube as discussed hereinabove. In this manner, internal lumen 66 of second member 62 is in fluid communication with one catheter lumen. The remaining catheter lumen is closed by virtue of the solid first member 60. Irrigation or aspiration may be carried out through second member 62 to appropriately irrigate or aspirate the selected catheter lumen of the catheter. Thereafter, the clinician may subsequently disconnect catheter adapter 100 from the dual lumen catheter and rotate adapter body 52 approximately 180° such that the second member 62 is aligned with the other catheter lumen. Subsequently, the clinician may supply pressure or vacuum to the other catheter lumen. Catheter adapter 100 may be devoid of a valve mechanism.

In the alternative, a one-way valve may be included in catheter adapter apparatus or may be externally connected thereto. The one-way valve permits fluid to be introduced into an attached dual lumen catheter or other connected device and inhibit reverse flow from the dual lumen catheter or connected device towards the pressure/vacuum source. By providing flow control between the pressure or vacuum source and the dual lumen catheter, the clinician is able to positively direct the pressure or vacuum to a selected lumen in an attached dual lumen catheter.

The catheter adapters of the present disclosure are contemplated for use in connection with priming, flushing, and/or aspiration of hemodialysis catheters during a medical procedure. For example, in one hemodialysis procedure, a catheter is installed through subcutaneous tunneling as disclosed in U.S. Pat. No. 4,832,687 to Smith, III and U.S. Pat. No. 5,944,732 to Raulerson, the entire contents of each of the '687 patent and the '732 patent being incorporated herein by reference.

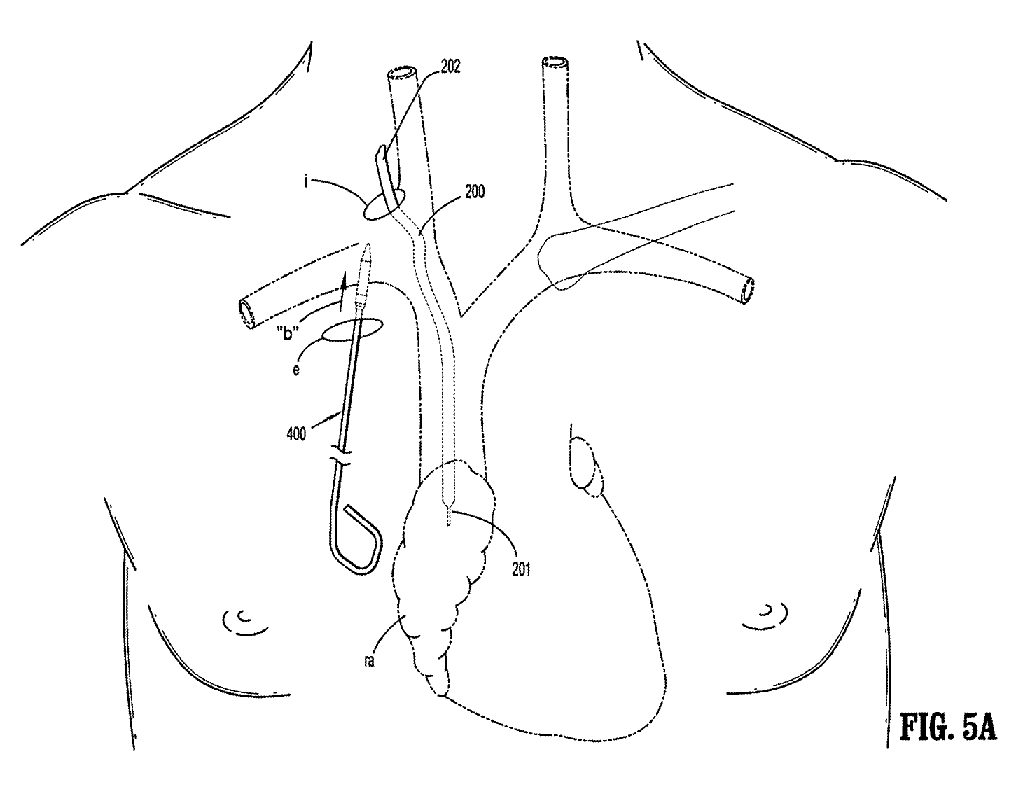

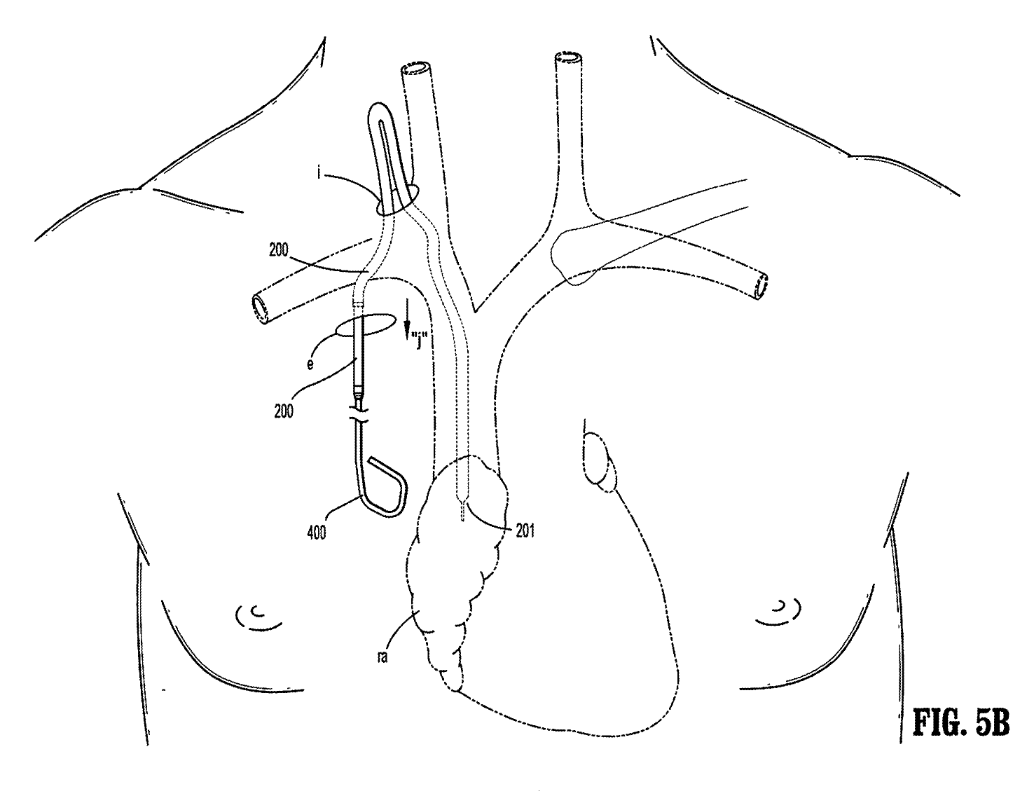

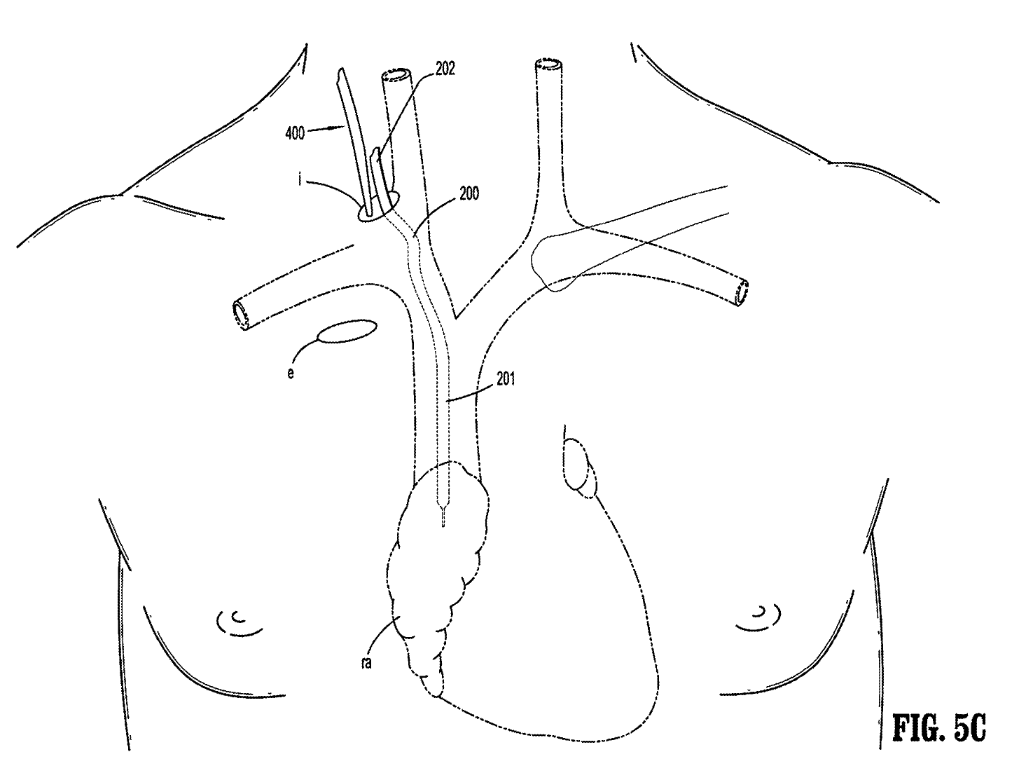

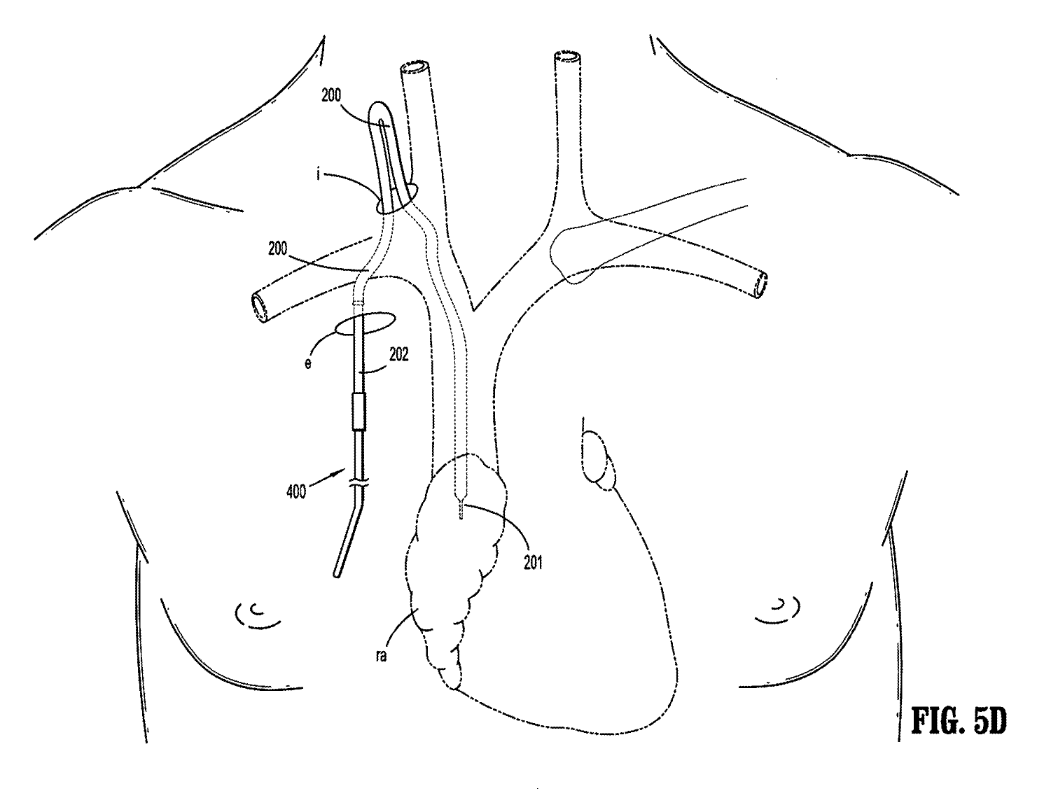

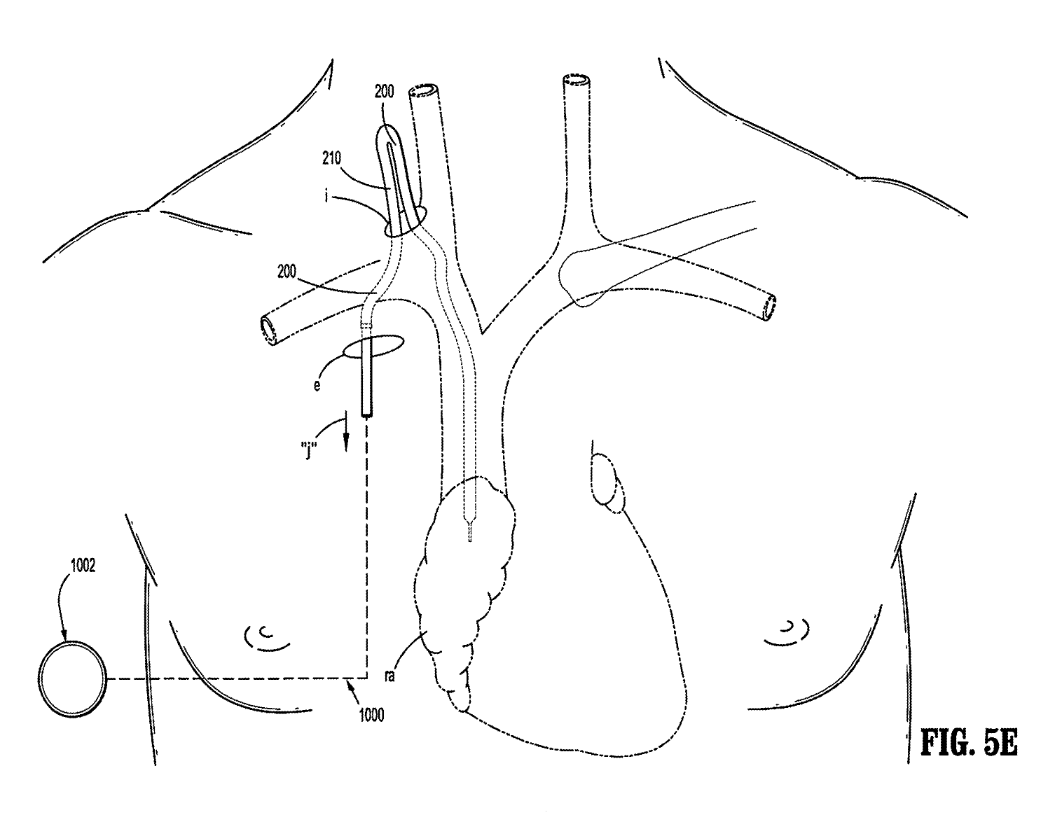

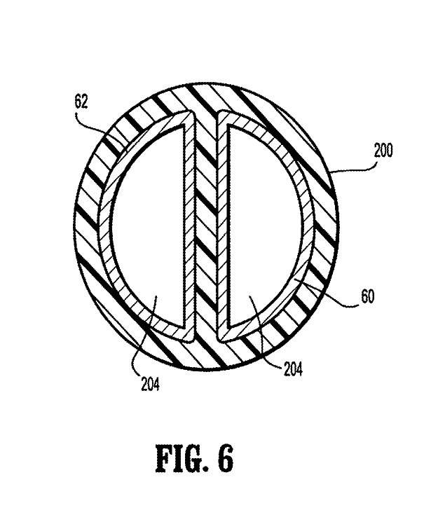

In one embodiment, the catheter may be implanted within a major vein of a patient via the reverse tunneling method disclosed in U.S. Pat. No. 5,509,897 to Twardowski, the entire contents of the '897 patent being incorporated herein by reference. In accordance with one embodiment of this procedure disclosed in the '897 patent and depicted in FIGS. 5A-5C, a catheter 200 is positioned through an incision “i” and advanced to enter the right atrium “ra”. To place catheter 200 in the right atrium “ra” the internal jugular vein is initially located and punctured with an introducer needle and a guidewire is inserted into the vessel using known techniques. The clinician makes an incision i” close to the path of the guide wire. The needle is removed, and the incision “i” from the skin to the vessel is enlarged adjacent to and along the pathway of the guidewire into the vessel so that a catheter may be inserted into the vessel. Before inserting the catheter 200 into the right atrium “ra”, the clinician connects the catheter adapter 50 to the catheter 200. In this regard, each of the first and second members 60, 62 of adapter body 52 is positioned within a respective catheter lumen 204 of the catheter, which may be, e.g., a dual lumen catheter as depicted in cross-section in FIG. 6. Preferably, first and second members 60, 62 form a frictional relationship with the internal surfaces defining the catheter lumens 204 of catheter 200. After properly securing the catheter adapter 50 to the trailing end 202 of the catheter 200, the clinician flushes, aspirates, or primes the lumens of the catheter 200 through the catheter adapter 50. The leading end 201 of a catheter 200 is advanced within the incision “i”, through the jugular vein, the superior vena cava, and into the right atrium “ra.” The clinician then may clamp the trailing end 202 of catheter 200. The catheter adapter 50 is removed from the trailing end 202 of the catheter 200 by either pulling the catheter adapter 50 from the catheter 200 or by cutting the catheter 200.

The clinician may make an exit opening “e” spaced apart from the initial incision “i.” The clinician advances a tunneling instrument 400 subcutaneously from exit opening “e” toward initial incision “i,” thereby creating a subcutaneous tunnel, as depicted in FIG. 5A. The tunneling instrument 400 is attached to the trailing end 202 of the catheter 200. Various mechanism for connecting the trailing end 202 of the catheter 200 to a tunneling instrument are envisioned including, e.g., with the use of a grasping mechanisms or the like. One suitable tunneling instrument is disclosed in commonly assigned U.S. patent application Ser. No. 11/986,861, filed Nov. 27, 2007 to Haarala, the contents and disclosure of which are hereby incorporated herein by its entirety. The clinician pulls the catheter 200, in the direction of arrow “j”, through the subcutaneous tunnel until the trailing end of the catheter 200 is exposed as shown in FIG. 5B.

Alternatively, in accordance with another tunneling procedure, a surgical tunnel is created between the initial incision “i” and to an exit site “e” remote from the original incision “I,” as illustrated in FIGS. 5C and 5D. Before making the subcutaneous tunnel, the clinician may connect the trailing end 202 of catheter 200 to a tunneling instrument 400. The clinician may then establish a subcutaneous tunnel by introducing the tunneling instrument 400 through initial incision “i” and advancing it toward exit site “e” until the trailing end 202 of the catheter 200 passes through exit site “e.” In addition to the explicitly disclosed methods, the clinician may employ any other suitable tunneling procedure.

Regardless of the tunneling methodology employed, once the trailing end 202 of catheter 200 is exposed as depicted in FIG. 5E, the clinician may optionally mount the catheter adapter 50 to trailing end 202 of the catheter tube 200 to perform priming, flushing, and aspiration functions. In this regard, each of the first and second members 60, 62 of adapter body 52 is positioned within a respective catheter lumen 204 of the catheter as depicted in cross-section in FIG. 6. Preferably, first and second members 60, 62 form a frictional relationship with the internal surfaces defining the catheter lumens 204 of catheter 200. Thereafter, flushing, aspiration, priming functions are performed as discussed hereinabove. Once the functions are completed, trailing end 202 of catheter 200 may be connected to a multiple tube connection assembly or catheter hub, which is in fluid communication with a hemodialysis machine to perform the hemodialysis procedure. Fluid coupling and the hemodialysis machine are shown schematically as reference numeral 1000, 1002 in FIG. 5E. It is also envisioned that the catheter adapter 50 may be mounted to the trailing end 202 of catheter 200 prior to the tunneling step to provide flushing or aspiration as necessary.

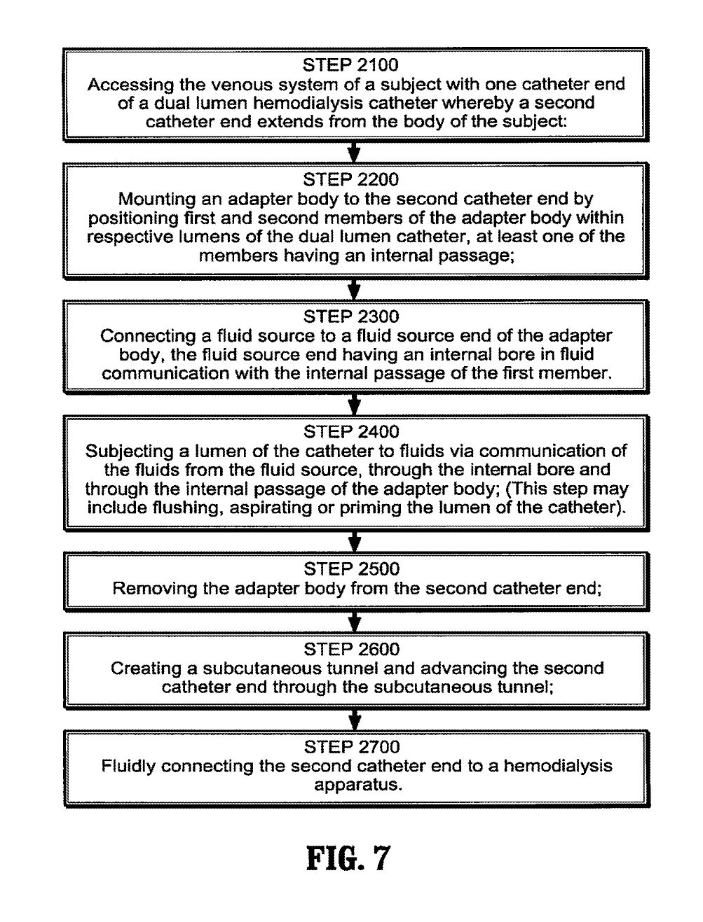

FIG. 7 generally illustrates a flow chart depicting a method of employing catheter adapter 50 for performing a hemodialysis procedure in accordance with the discussion hereinabove. The method 2000 may include the steps of (step 2100) accessing the venomous system of a subject with one catheter end of a dual lumen hemodialysis catheter whereby a second catheter end extends from the body of the subject, (step 2200) mounting an adapter body to the second catheter end by positioning first and second members of the adapter body within respective lumens of the dual lumen, at least one of the members having an internal passage; (step 2300) connecting a fluid source to a fluid source end of the adapter body, the fluid source end having an internal bore in fluid communication with the internal passage of the first member; (step 2400) subjecting a lumen of the catheter to fluids via communication of the fluids from the fluid source, through the internal bore and through the internal passage of the adapter body (step 2400 may include flushing, aspirating, or priming the lumens of the catheter.); (step 2500) removing the adapter body from the second catheter end; (step 2600) creating a subcutaneous tunnel and advancing the second catheter end though the subcutaneous tunnel; and (step 2700) fluidly connecting the second catheter end to a hemodialysis machine.

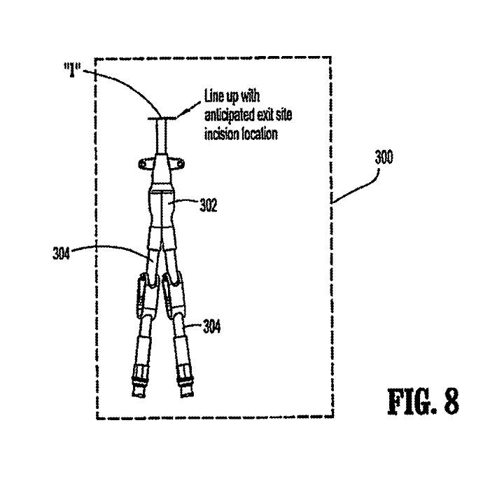

A further feature of the present invention is the provision of a template 300 to facilitate the placement of the catheter hub and/or extensions for securement to the catheter 200 during the aforementioned reverse tunneling procedures. With reference to FIG. 8, template 300 is preferably a card or transparent film having an image of the catheter hub 302 and extensions 304, which may be generally similar to the catheter hub and extensions to be subsequently attached to proximal end 202 of catheter 200 after the reverse tunneling procedure. In use, the anticipated exit site “e” is identified within the chest area downward of the venotomy or initial incision “i”. The exit site “e” optionally may be formed within the chest area. The simulated exit site line “l” of template 300 is then generally aligned with the proposed or actual exit site “e” incision thereby positioning the simulated catheter hub 302 and extensions 304 of the template 300 further downward from the exit site “e” as shown. This positioning corresponds to a desired position or location of the actual catheter hub to be connected to the proximal end of the catheter. Thus, the clinician may position the catheter hub on the template 300 and secure the catheter hub to the patient with surgical tape or the like. Once the proximal end 202 of the catheter 200 is exposed from the exit site “e” after the reverse tunneling procedure, the proximal end 202 of the catheter 200 may be readily connected to the catheter hub 302.

It will be understood that various modifications may be made to the embodiments of the presently disclosed catheter adapter apparatus. Therefore, the above description should not be construed as limiting, but merely as exemplifications of embodiments. Those skilled in the art will envision other modifications within the scope and spirit of the present disclosure.

1-12. (canceled)

13. A catheter apparatus comprising:
an adapter body having a fluid source end adapted for connection to a fluid source and a catheter end adapted for connection to a catheter tube, the catheter end including first and second members extending therefrom, each of the first and second members being adapted for reception within corresponding lumens of the catheter tube, the first member defining an internal bore and the second member having a substantially solid interior.

14. The catheter adapter apparatus of claim 13, wherein the fluid source end defines an internal bore in fluid communication with the internal bore of the first member.

15. The catheter adapter apparatus of claim 13, wherein the fluid source end includes a luer connector.

16. The catheter adapter apparatus of claim 13, further comprising a valve member operatively associated with the adapter body, the valve member being adapted to selectively control fluid flow between the internal bore of the fluid source end and the internal bore of the first member.

17. A surgical method for a hemodialysis procedure, the method comprising:
accessing the venous system of a subject with one catheter end of a dual lumen hemodialysis catheter whereby a second catheter end of the catheter extends from the body of the subject;
mounting an adapter body to the second catheter end by positioning first and second members of the adapter body within respective lumens of the dual lumen catheter, at least one of the members having an internal passage;
connecting a fluid source to a fluid source end of the adapter body, the fluid source end having an internal bore in fluid communication with the internal passage of the first member;
subjecting a lumen of the catheter to fluids via communication of the fluids from the fluid source, through the internal bore and through the internal passage of the adapter body;
removing the adapter body from the second catheter end; and
fluidly connecting the second catheter end to a hemodialysis apparatus.

18. The surgical method according to claim 17, including the step of creating a subcutaneous tunnel and advancing the second catheter end through the subcutaneous tunnel.

19. The surgical method according to claim 18, wherein the step of subjecting includes one of flushing, aspirating or priming of the lumen of the catheter.

20. The surgical method according to claim 17, wherein the first member defines the internal bore and the second member has a substantially solid interior.

21. The surgical method according to claim 20, wherein mounting the adapter body to the second catheter end comprises positioning the first member of the adapter body within a first lumen of the catheter and positioning the second member of the adapter body within a second lumen of the catheter.

22. The surgical method according to claim 21, the method further comprising:
after removing the adapter body from the second catheter end, rotating the adapter body and mounting the adapter body to the second catheter end by positioning the first member of the adapter body within the second lumen of the catheter and positioning the second member of the adapter body within the first lumen of the catheter; and
subjecting the second lumen of the catheter to fluids via communication of the fluids from the fluid source, through the internal bore and through the internal passage of the adapter body.
